(19) United States
(12) Patent Application Publication
(10) Pub. No.: US 2020/0345905 A1
(43) Pub. Date: Nov. 5, 2020

(54) SURGICAL SMOKE REMOVING DEVICE

(71) Applicant: National Cheng Kung University, Tainan City (TW)

(72) Inventors: Ming-Yeng LIN, Kaohsiung City (TW); Huann-Shyang LIN, Tainan City (TW); Kun-Chan LAN, Tainan City (TW); Yao-Lung KUO, Tainan City (TW)

(21) Appl. No.: 16/861,265

(22) Filed: Apr. 29, 2020

Related U.S. Application Data
(60) Provisional application No. 62/841,833, filed on May 2, 2019.

(51) Int. Cl. A61M 1/00; B01D 46/44; A61B 18/14

(52) U.S. Cl. CPC A61M 1/0066; B01D 46/44; A61B 18/14; A61M 2209/01; A61B 2218/008; A61M 2205/3375; A61M 2205/3306; A61M 2205/75; A61M 2205/3303

(57) ABSTRACT

The present invention provides a surgical smoke removing device, which includes a smoke removing apparatus adapted to be connected to an application apparatus. The smoke removing apparatus includes an air cleaner, a stage, and a controller. The air cleaner has an inlet to suck in air, and an outlet to blow out clean air. The stage is configured to move the outlet of the air cleaner, such that the outlet targets at an emission source and/or a breathing zone of a user. The controller is connected to the air cleaner and the stage.

### CROSS REFERENCE TO RELATED APPLICATION

This application claims the benefit of filing date of U.S. Provisional Application Ser. No. 62/841,833, entitled “AN APPLIANCE WITH AIR POLLUTION REDUCTION DEVICE” filed May 2, 2019 under 35 USC § 119(e)(1).

### BACKGROUND OF THE INVENTION

### 1. Field of the Invention

The present invention relates to an air pollution reduction device and, more particularly, to a surgical smoke removing device.

### 2. Description of Related Art

Air pollution (including air pollutants or polluted air) can be generated from human activities such as cooking, surgery, smoking, drilling, or welding. In particular, the use of an electrosurgical pencil in an operating room, the use of a dental drill in a dental clinic, or the use of a welding torch in welding factory can generate noticeable air pollution.

In the case of the operating room, the electrosurgical pencil is often used for cutting, coagulating, desiccating, or fulgurating tissue. The surgical smoke is often produced during the surgery process using the electrosurgical pencil. The surgical smoke contains harmful substances, such as volatile organic compounds (VOCs), particulate matter (PM), bioaerosols, etc. Researches have shown that the surgical smoke may cause headaches, allergies, or airborne diseases (see references 1 and 2 listed below). In particular, surgeons or medical personnel involved in the surgery may suffer from respiratory diseases due to inhalation of surgical smoke. Therefore, the surgical smoke produced by the electrosurgical pencil can be harmful and should not be ignored.

One type of air pollution reduction device is a face-wearing device (e.g. a face mask), which reduces the possibility of inhaling the air pollution. However, some users may feel uncomfortable for they have to wear additional objects that cover their faces. Another type of air pollution reduction device is an air cleaner (machine), which may further be divided into two types: the stand-alone type and the portable type.

The stand-alone air cleaner is a device on its own (which includes a fixed purification base and a movable suction tube to be held near the operation place) and not installed onto the electrosurgical pencil. The base cannot be moved around easily. In addition, such stand-alone air cleaner is often operated by a person who needs to hold the suction tube near the operation location. Using the stand-alone air cleaner will increase labor costs, and more people will expose to the air pollution.

As for the portable air cleaner, it can be installed on the electrosurgical pencil. The inlet of the portable air cleaner is usually designed to be close to the tip of the electrosurgical pencil in order to capture the surgical smoke. However, the portable air purifier has only one single inlet that can only capture the surgical smoke in a small area. Meanwhile, the electrosurgical pencil attached with the portable air cleaner will take up more volume, and thus is inconvenient for the medical personnel to handle.

In the case of the dental clinic, the dental drill operated during the dental treatment will emit bioaerosols or particulate matter. Many current clinics only use stand-alone suction devices to remove air pollution generated during dental procedure. However, a stand-alone suction device may obstruct the dentist's sight, and thus affect the dental treatment. In addition, the stand-alone suction device cannot provide enough protection to the medical personnel. Therefore, there is still a need to improve the air pollution removal device in the dental clinic.

In the case of the welding factory, a welding process will produce a great amount of air pollution. Researches have shown that welding workers often suffer from lung cancer and other respiratory diseases (see references 3 and 4 listed below). The welding factory commonly uses a local exhaust ventilation (LEV) to remove the air pollution. However, it may obscure the operator's field of vision and operation.

Therefore, it is desirable to provide an improved electronic apparatus to mitigate and/or obviate the aforementioned problems.

### REFERENCES ARE LISTED AS FOLLOWS

1. Pierce, J. S.; Lacey, S. E.; Lippert, J. F.; Lopez, R.; Franke, J. E., Laser-Generated Air Contaminants from Medical Laser Applications: A State-of-the-Science Review of Exposure Characterization, Health Effects, and Control. Journal of Occupational and Environmental Hygiene 2011, 8, (7), 447-466.
2. Okoshi, K.; Kobayashi, K.; Kinoshita, K.; Tomizawa, Y.; Hasegawa, S.; Sakai, Y., Health risks associated with exposure to surgical smoke for surgeons and operation room personnel. Surgery today 2015, 45, (8), 957-965.
3. MacLeod, J. S.; Harris, M. A.; Tjepkema, M.; Peters, P. A.; Demers, P. A., Cancer Risks among Welders and Occasional Welders in a National Population-Based Cohort Study: Canadian Census Health and Environmental Cohort. Saf. Health Work 2017, 8, (3), 258-266.
4. Antonini, J. M.; Lewis, A. B.; Roberts, J. R.; Whaley, D. A., Pulmonary effects of welding fumes: Review of worker and experimental animal studies. Am. J. Ind. Med. 2003, 43, (4), 350-360.

### SUMMARY OF THE INVENTION

The surgical smoke removing device of the present invention can effectively reduce the air pollution concentration of the nearby pollution source and/or deliver clean air to the user's breathing zone. The user can choose to direct the airflow to the nearby pollution source and/or the breathing zone. In other embodiments, the user can also choose to suck air pollution from the emission source and/or deliver clean air to the user's breathing zone.

In view of this, according to one aspect of the present invention, there is provided a surgical smoke removing device including a smoke removing apparatus. The smoke removing apparatus is adapted to be connected to an application apparatus. The smoke removing apparatus includes an air cleaner, a stage, a tracer, and a controller. The air cleaner has an inlet to suck air in and an outlet to blow clean air out. The stage is configured to move the outlet of the air cleaner, such that the outlet targets at an emission source and/or a user's breathing zone. The tracer is configured to trace (or track) the emission source and/or the user's breathing zone, and send a sensed signal to the controller or the stage. The controller is connected to the air cleaner, the stage, the tracer, and/or a power supplier.

Optionally or preferably, the application apparatus is a surgical lighting apparatus or a head lamp, which has a front side. The front side faces the emission source and/or the user's breathing zone. The outlet of the air cleaner of the smoke removing apparatus is formed as a hole or a protrusion on the front side.

Optionally or preferably, the tracer is an image recognizer, a motion sensor, or a displacement sensor, configured to trace a tip of a tool or a user's face, hand, surgical incision or anatomical sites, or other target parts; or alternatively, the tracer is a sound sensor or an ultrasound sensor, configured to detect the existence of living being; or alternatively, the tracer is an infrared sensor, an optical sensor, or a visible light sensor, configured to trace a hot spot of a tool; or alternatively, the tracer is a mass sensor, a chemosensor (chemical sensor), a molecular sensor, an electronic nose, a gas chromatograph, or a mass spectrometer, configured to detect the existence, the component the concentration of polluted air.

Optionally or preferably, in the following embodiments of the present invention, the tracer may be configured to trace any or any combinations of the following terms: (i) a physical term, such as position, distance, movement, displacement, speed, acceleration, color, sound, smell, inclination, roughness, mass, etc.; (ii) a target point, such as air pollution emission source, high concentration zone of air pollution, air pollution hot point, human face, human hand, other target parts, etc.; (iii) an object, such as scalpel, cigarette, electronic cigarette, fire candle, etc.

Optionally or preferably, the air cleaner is a suction pump, a blower, or a fan.

Optionally or preferably, the air cleaner includes a filter component. The filter component may be a high efficiency particulate air (HEPA) filter, an ultra-low penetration air (ULPA) filter, a membrane filter, a nanofiber filter, an active carbon filter, a gas filter, a catalyst filter, an antimicrobial filter, an ionizer, an ultraviolet air purifier with ultraviolet light source, an ozone generator, an impactor, or a cyclone, or combination thereof.

Optionally or preferably, the air cleaner includes an air compressor or a suction device; or alternatively, the air cleaner is connected to an external air compressor or an external suction device that can provide the air cleaner with airflow.

Optionally or preferably, the stage is an XYZ translation stage, a translation stage, a two-axis stage, a rotary stage, a tilt stage, a ball-and-socket tilt stage, a mechanical arm, or any stage that is capable of carrying a moving object.

Optionally or preferably, the air cleaner further includes an additional outlet. The outlet and additional outlet are both configured to blow purified air out or suck polluted air in, or one outlet is configured to blow purified air out while another outlet is configured to suck polluted air in.

Optionally or preferably, the outlet and the additional outlet are arranged such that they are both adjacent to the application apparatus, or one is adjacent to the application apparatus and another is far away from the application apparatus.

Optionally or preferably, the additional outlet is formed by a plurality of holes which blow air out to form the air curtain, and the air curtain is located between the emission source and the user's breathing zone.

Optionally or preferably, the controller is configured to control the application apparatus, the air cleaner (so as to turn it on/off or switch between a suction mode and a blow mode, or control the amount of the airflow), the stage (so as to move the stage), the tracer, the power supplier, or an external apparatus (such as an air compressor, a suction device, or an individual air cleaner). The controller is connected to the component which it aims to control. Moreover, the controller may be configured to communicate with, control, or be controlled by one or more of the following devices, such as the main body of the application apparatus (for example, the power supplier of the surgical lighting apparatus), a personal computer, a laptop, a tablet, a smartphone, or a remote controller.

According to another aspect of the present invention, there is provided an air pollution reduction device including an application apparatus and a smoke removing apparatus. The application apparatus is a range hood, a frying pan, a wok, a pot, a cooker, a tea kettle, a coffeemaker, a popcorn maker, a necklace, a pair of glasses, a wristband, a watch, a mask, or an earring. The smoke removing apparatus is connected to the application apparatus. The smoke removing apparatus includes an air cleaner, a stage, and a controller. The air cleaner has an inlet to suck air in and an outlet to blow clean air out. The stage is configured to move the outlet of the air cleaner, such that the outlet targets at an emission source and/or a user's breathing zone. The controller is connected to the air cleaner and the stage.

According to another aspect of the present invention, there is provided a surgical smoke removing method, comprising: Step S1 is turning on a surgical smoke removing device during a surgery period; wherein the surgical smoke removing device includes an application apparatus and a smoke removing apparatus; the smoke removing apparatus is connected to the application apparatus; the smoke removing apparatus includes an air cleaner, a stage, and a controller; the controller is connected to the air cleaner and the stage. Step S2 is using the stage to move the air cleaner, such that an outlet of the air cleaner targets at an emission source and/or a user's breathing zone. Step S3 is using the air cleaner to blow purified air from the outlet.

Optionally or preferably, the surgical smoke removing device further includes a tracer. The method further includes using the tracer to trace the emission source and/or the user's breathing zone, and send a sensed signal to the controller or the stage.

Other objects, advantages, and novel features of the invention will become more apparent from the following detailed description when taken in conjunction with the accompanying drawings.

### BRIEF DESCRIPTION OF THE DRAWINGS

FIGS. 1 and 2 show a schematically structural diagram and a schematically application diagram of the surgical smoke removing device according to one embodiment of the present invention, respectively;

FIG. 3 is a variant of FIG. 1;

FIG. 4 is a variant of FIG. 1;

FIG. 5 is another variant of FIGS. 1 and 2;

FIG. 6 is still another variant of FIGS. 1 and 2;

FIG. 7 shows a schematically structural diagram of the surgical smoke removing device according to one embodiment of the present invention;

FIG. 8 is a variant of FIG. 7;

FIG. 9 shows a schematically structural diagram of the air pollution reduction device (which may be considered to be a general surgical smoke removing device) according to one embodiment of the present invention;

FIG. 10 shows a schematically structural diagram of the air pollution reduction device (which may be considered to be a general surgical smoke removing device) according to one embodiment of the present invention;

FIG. 11 is a variant of FIG. 10;

FIG. 12 shows a schematically structural diagram of the air pollution reduction device (which may be considered to be a general surgical smoke removing device) according to one embodiment of the present invention.

### DETAILED DESCRIPTION OF THE EMBODIMENT

Different embodiments of the present invention are provided in the following description. These embodiments are meant to explain the technical content of the present invention, but not meant to limit the scope of the present invention. A feature described in an embodiment may be applied to other embodiments by suitable modification, substitution, combination, or separation.

It should be noted that, in the present specification, when a component is described to have an element, it means that the component may have one or more of the elements, and it does not mean that the component has only one of the element, except otherwise specified.

Moreover, in the present specification, the ordinal numbers, such as “first” or “second”, are used to distinguish a plurality of elements having the same name, and it does not means that there is essentially a level, a rank, an executing order, or an manufacturing order among the elements, except otherwise specified. A “first” element and a “second” element may exist together in the same component, or alternatively, they may exist in different components, respectively. The existence of an element described by a greater ordinal number does not essentially means the existence of another element described by a smaller ordinal number.

In the present specification, a feature A “or” or “and/or” a feature B means that the feature A may exist alone, the feature B may exist alone, or the features A and B may exist together; while, a feature A “and” a feature B means that the features A and B exist together; the terms “include”, “comprise”, “contain”, or “have” means “include but is not limited there to”, except otherwise specified.

Moreover, in the present specification, the terms, such as “top”, “bottom”, “left”, “right”, “front”, “back”, or “middle”, as well as the terms, such as “on”, “above”, “under”, “below”, or “between”, are used to describe the relative positions among a plurality of elements, and the described relative positions may be interpreted to include their translation, rotation, or reflection.

Moreover, in the present specification, when an element is described to be arranged “on” another element, it does not essentially means that the elements contact the other element, except otherwise specified. Such interpretation is applied to other cases similar to the case of “on”.

Moreover, in the present specification, the terms, such as “preferably” or “advantageously”, are used to describe an optional or additional element or feature, and in other words, the element or the feature is not an essential element, and may be ignored in some embodiments.

Moreover, in the present specification, when an element is described to be “suitable for” or “adapted to” another element, the other element is an example or a reference helpful in imagination of properties or applications of the element, and the other element is not to be considered to form a part of a claimed subject matter.

[Embodiment Relevant to Surgical Lighting Apparatus]

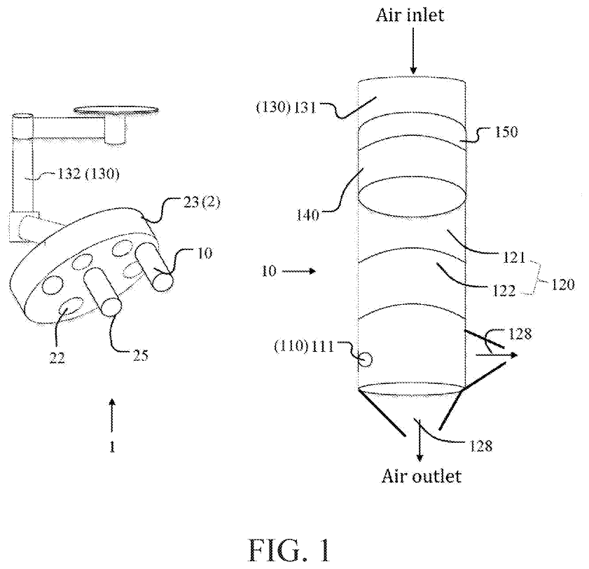

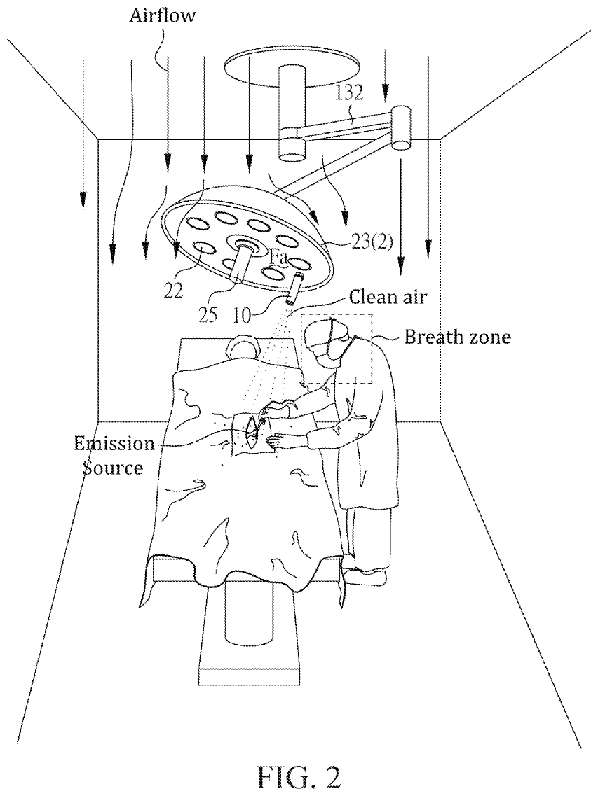

FIGS. 1 and 2 show a schematically structural diagram and a schematically application diagram of the surgical smoke removing device 1 according to one embodiment of the present invention, respectively.

As shown in FIGS. 1 and 2, the air pollution reduction device 1 of the present invention includes an application apparatus 2 and a smoke removing apparatus 10. In this example, the application apparatus 2 is a surgical lighting apparatus 23, but not limited thereto. The surgical lighting apparatus 23 is typically ceiling mounted. The surgical lighting apparatus 23 has a handle 25, located at the center of its front side Fa, which is convenient for medical personnel to grasp and move the surgical lighting apparatus 23. The smoke removing apparatus 10 may be installed on the surgical lighting apparatus 23, in particular on its front side Fa, and near a lamp 22 of the surgical lighting apparatus 23. Preferably, the smoke removing apparatus 10 is installed on the surgical lighting apparatus 23 and is detachable.

In an operation room, clean laminar airflow will flow down from a ceiling exhaust; it can press various pollutants toward the ground, and further toward the ground exhaust. However, during the surgery process, the surgical lighting apparatus 23 often blocks the laminar flow that flows down from the ceiling, so the person under the surgical lighting apparatus 23 may not obtain sufficient protection from the ceiling laminar flow. Commonly, the person under the surgical lighting apparatus 23 is also the one closest to the pollution source, and needs more protection. In addition, the electrocautery surgery usually generates a great amount of air pollution from the electrocautery location, and needs to be removed efficiently.

The tracer 110 is configured to trace a target point or a surgical site near the tip of an electrosurgical pencil during the surgery process, by using image recognition technology. The tracer can also trace the user's face, hand, or other target parts. In other embodiments, the tracer 110 may be an infrared sensor 111 to trace a location or a hot spot when the electrosurgical pencil is being used. The tracer 110 will accordingly generate a sensed signal, and send the sensed signal to the controller 140.

Then, the controller 140 will send a control signal to the stage 130 for the stage 130 to move the outlet 128 of the air cleaner 120, such that the outlet 128 targets at the emitting gas and/or the user. The stage 130 may be an XYZ translation stage 131 or a mechanical arm 132. When the mechanical arm 132 is introduced to serve as the stage 130, the mechanical arm 132 may further be configured to automatically move the entire surgical lighting apparatus 23 to face the operation zone (e.g. the surgical site) or the zone required by the user (e.g. the surgeon), according to the sensed signal. In this way, it is not necessary to move the surgical lighting apparatus 23 manually by the user's hand (the action may take time and the user's hand may be contaminated with blood), and thus it can save time and reduce the pollution of the handle 25. Meanwhile, since the automation of the surgical lighting apparatus 23 can increase light brightness, the tracer 130 can more accurately trace the target point by using image recognition or other technologies.

The air cleaner 120 may be a suction pump, a blower, or a fan 121, and may optionally be provided with any of the aforementioned filter components 122 or their combinations. The air cleaner 120 has an inlet 127 to suck air in. The inlet 127 may suck polluted air in by itself, or alternatively, the inlet 127 may be connected to an external apparatus (e.g. a heating, ventilation, and air conditioning (HVAC) or an external air source) that can provide purified air. The air cleaner 120 also has at least one outlet 128 (FIG. 1 shows two, but not limited thereto) to blow clean air out. In a suction mode, the air cleaner 120 may suck polluted air in and filter out the polluted air. In a blow mode, the air cleaner 120 may blow purified (clean) air toward the emission source, so as to dilute the air pollution concentration at the source.

The power supplier 150 is used to supply power to the application apparatus 2 and/or the smoke removing apparatus 10 (including components therein, such as an air cleaner) of the air pollution reduction device 1.

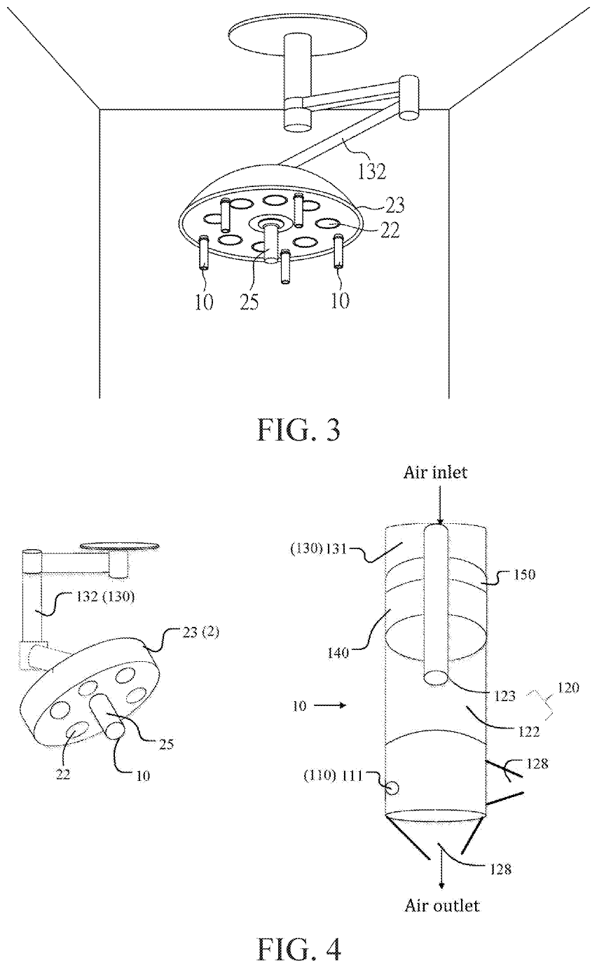

FIG. 3 is a variant of FIG. 1.

In the example of FIG. 3, a plurality of lamps 22 and a plurality of smoke removing apparatuses 10 are arranged interspersed with each other.

FIG. 4 is a variant of FIG. 1.

As shown in FIG. 4, the air cleaner 120 may include an air compressor 123, so as to facilitate or replace the suction pump, the blower, or the fan 121. Besides, in the example of FIG. 4, the smoke removing apparatus 10 is alternatively installed near the handle 25 of the surgical lighting apparatus 23; it may even be embedded into the handle 25.

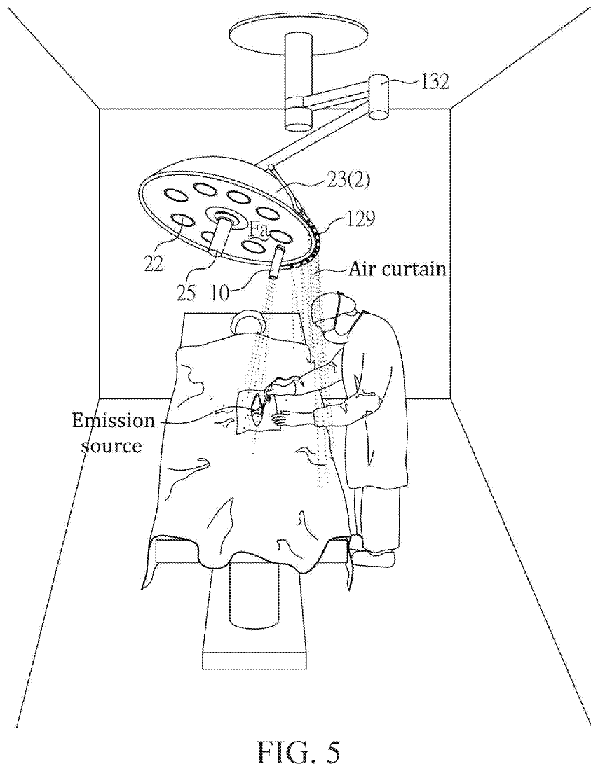

FIG. 5 is another variant of FIGS. 1 and 2.

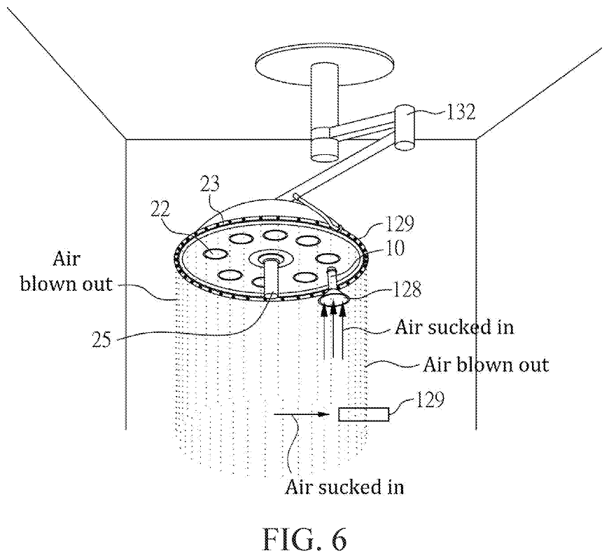

As shown in FIG. 5, the air cleaner 120 is formed with the outlet 128, and also formed with one or more additional outlets 129. In another interpretation, the additional outlet 129 may be formed by a plurality of holes. The additional outlet 129 may blow air out to form an air curtain. The outlet 128 and the additional outlet 129 may be arranged such that they are both adjacent to the application apparatus 2 (for example, installed thereon), or one is adjacent to the application apparatus 2 (for example, installed thereon) and another is far away from the application apparatus 2 (for example, arranged on a surgical table, the ground, a bed, a wall, or another independent apparatus, as shown in FIG. 6).

In the example of FIG. 5, the additional outlet 129 is arranged at the edge of the surgical lighting apparatus 23, in particular arranged on the side near the user (e.g. the surgeon), in particular arranged in a half circle at the edge of the surgical lightening apparatus (assuming that the surgical lightening apparatus has round shape). Such arrangement is advantageous, because the air curtain formed by the air blown out from the additional outlet 129 will be located between the emission source and the user's breathing zone, and thus protect the user. In addition, in the blow mode, when the air cleaner 120 blows purified (clean) air toward the emission source, so as to dilute polluted air at the source, the air curtain can avoid polluted air flowing toward the user, thereby giving the user even more protection.

FIG. 6 is still another variant of FIGS. 1 and 2.

In the example of FIG. 6, the additional outlet 129 is arranged at the edge of the surgical lighting apparatus 23, and arranged in one circle. Such arrangement is advantageous, because the air curtain formed by the air blown out from the additional outlet 129 will be located between the emission source and the user's breathing zone, and the pollutants will be surrounded (or contained) by the air curtain, and thus protect the user, including the surgeon or the other medical personnel. When the additional outlet 129 is in the blow mode, the outlet 128 may be set to be in the suction mode, to remove polluted air surrounded by the air curtain formed by the air blown out from the additional outlet 129. The outlet 128 may also be independently located far away from the additional outlet 129 (but for example, near the patient or the medical personnel).

Besides, when the additional outlet 129 is in the blow mode, and the outlet 128 is also in the blow mode, the air curtain can avoid polluted air flowing back to the user, thereby giving the user even more protection.

[Embodiment Relevant to Head Lamp]

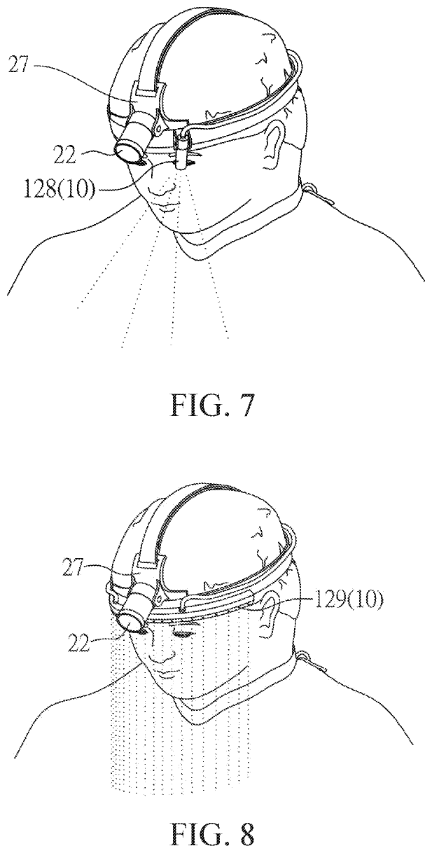

FIG. 7 shows a schematically structural diagram of the surgical smoke removing device 1 according to one embodiment of the present invention.

As shown in FIG. 7, the air pollution reduction device 1 of the present invention includes an application apparatus 2 and a smoke removing apparatus 10. In this example, the application apparatus 2 is a head lamp 27 worn on a surgeon's head, but not limited thereto. The smoke removing apparatus 10 may be installed on the head lamp 27, in particular on its front side, and near the lamp 22 of the head lamp 27. Preferably, the smoke removing apparatus 10 is installed on the head lamp 27 and is detachable. In the blow mode, the air cleaner (not shown, but similar to those in other embodiments) of the smoke removing apparatus 10 may blows purified (clean) air from the outlet 128 toward the emission source, so as to dilute the concentration of polluted air at the source.

FIG. 8 is a variant of FIG. 7.

In the example of FIG. 8, the air cleaner is formed with the outlet 128 (referring to FIG. 7, for example), and also formed with one or more additional outlets 129. In another interpretation, the additional outlet 129 may be formed by a plurality of holes. The additional outlet 129 may blow air out to form an air curtain. The additional outlet 129 is arranged in front of the head lamp 27, in particular arranged in a half circle. Such arrangement is advantageous, because the air curtain formed by the air blown out from the additional outlet 129 will be located between the emission source and the user's breathing zone, and thus protect the user. Besides, in the blow mode, when the air cleaner blows purified (clean) air from the outlet 128 (referring to FIG. 7, for example) toward the emission source, so as to dilute polluted air at the source, the air curtain can avoid polluted air flowing towards the user, thereby giving the user even more protection.

[Embodiment Relevant to Range Hood]

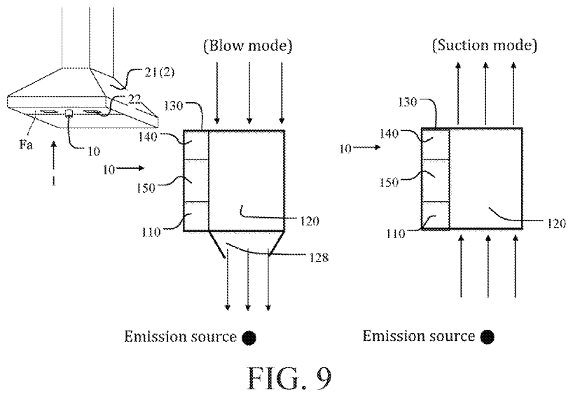

FIG. 9 shows a schematically structural diagram of the air pollution reduction device 1 (which may be considered to be a general surgical smoke removing device 1) according to one embodiment of the present invention.

As shown in FIG. 9, the air pollution reduction device 1 of the present invention includes an application apparatus 2 and a smoke removing apparatus 10. In this example, the application apparatus 2 is a range hood 21, but not limited thereto. The smoke removing apparatus 10 may be installed on the application apparatus 2, in particular on its front side Fa, and near the lamp 22 of the application apparatus 2. Preferably, the smoke removing apparatus 10 is installed on the application apparatus 2 and is detachable. Or optionally, the smoke removing apparatus 10 is embedded into the application apparatus 2.

The tracer 110 is configured to trace the residual emission gas and/or the emission gas approaching the user that the range hood 21 fails to capture, by using image recognition technology, for example. It is of course possible to trace the user's face, hand, or other target parts. Then, the tracer 110 will accordingly generate a sensed signal, and send the sensed signal to the controller 140.

Then, the controller 140 will send a control signal to the stage 130 for the stage 130 to move the outlet 128 of the air cleaner 120, such that the outlet 128 targets at residual emitting gas and/or the user. The stage 130 may be a ball-and-socket tilt stage or an XYZ translation stage.

The air cleaner 120 may be a suction pump, a blower, or a fan, and may optionally be provided with any of the aforementioned filter components or their combinations. In a scenario of an operation room, since the operation room's air inlet can provide very clean air, it is not necessary to equip any filter component into the air cleaner 120. In a suction mode, the air cleaner 120 may suck polluted air in and filter out the polluted air. In a blow mode, the air cleaner 120 may blow purified (clean) air toward the emission source, so as to dilute and/or contain the air pollution concentration at the source.

The power supplier 150 is used to supply power to the application apparatus 2 and the smoke removing apparatus 10 (including components therein, such as an air cleaner) of the air pollution reduction device 1.

[Embodiment Relevant to Frying Pan]

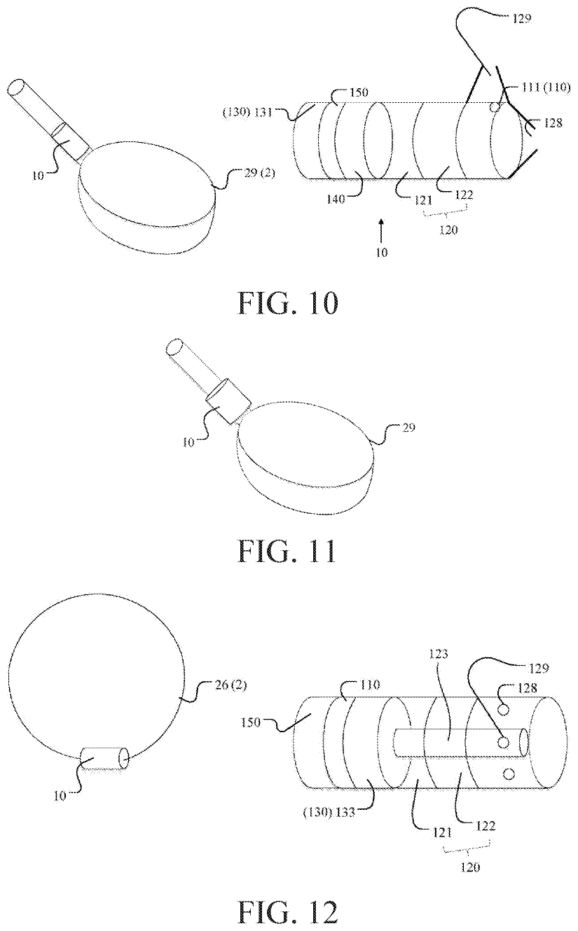

FIG. 10 shows a schematically structural diagram of the air pollution reduction device 1 (which may be considered to be a general surgical smoke removing device 1) according to one embodiment of the present invention.

FIG. 11 is a variant of FIG. 10.

As shown in FIG. 10, the air pollution reduction device 1 of the present invention includes an application apparatus 2 and a smoke removing apparatus 10. In this example, the application apparatus 2 is a flying pan 29; while, in other examples, the application apparatus 2 may be a wok, a pot, a cooker, a tea kettle, a coffeemaker, or a popcorn maker, but not limited thereto.

The smoke removing apparatus 10 may be installed on the application apparatus 2, in particular attached on its handle. In further particular, the extending direction of the channel the smoke removing apparatus 10 may be parallel to the extending direction of the handle of the application apparatus 2. Optionally, the smoke removing apparatus 10 is embedded into the application apparatus 2, as shown in FIG. 10. Or preferably, the smoke removing apparatus 10 is installed on the application apparatus 2 and is detachable, as shown in FIG. 11.

The tracer 110 is configured to trace the smoke or the steam emitted from the flying pan 29, by using image recognition technology, for example. It is of course possible to trace the user's face, hand, or other target parts. In other embodiments, the tracer 110 may be an infrared sensor 111 to trace a hot spot when the frying pan is being used. Then, the tracer 110 will accordingly generate a sensed signal, and send the sensed signal to the controller 140.

Then, the controller 140 will send a control signal to the stage 130 for the stage 130 to move the outlet 128 of the air cleaner 120, such that the outlet 128 targets at residual emitting (polluted) air and/or the user. The stage 130 may be a ball-and-socket tilt stage or an XYZ translation stage 131.

The air cleaner 120 may be a suction pump, a blower, or a fan 121, and may optionally be provided with any of the aforementioned filter components 122 or their combinations.

Moreover, the air cleaner 120 is formed with the outlet 128, and also formed with one or more additional outlets 129. In one embodiment, the outlet 128 and the additional outlet 129 are both set to blow purified (clean) air out, respectively toward the pollution emission source (in this case, the frying pan) and the user, so as to blow polluted air away. In another embodiment, the outlet 128 is configured to face the pollution emission source (in this case, the frying pan), to suck polluted air in and filter out the polluted air, while, the additional outlet 129 is configured to blow purified (clean) air to the user.

The power supplier 150 is used to supply power to the application apparatus 2 and the smoke removing apparatus 10 (including components therein, such as an air cleaner) of the air pollution reduction device 1.

[Embodiment Relevant to Necklace]

FIG. 12 shows a schematically structural diagram of the air pollution reduction device 1 (which may be considered to be a general surgical smoke removing device 1) according to one embodiment of the present invention.

As shown in FIG. 12, the air pollution reduction device 1 of the present invention includes an application apparatus 2 and a smoke removing apparatus 10. In this example, the application apparatus 2 is a necklace 26; while, in other examples, the application apparatus 2 may be a wearable device, such as a pair of glasses, a wristband, a watch, a mask, or an earring, but not limited thereto.

The smoke removing apparatus 10 may be installed on the application apparatus 2, in particular attached on the lace of the necklace. (Correspondingly, the smoke removing apparatus 10 may be attached on the glasses frame, the band, the watchband, the mask surface, or the earring surface in other embodiments). Optionally, the smoke removing apparatus is embedded into the application apparatus 2. Or preferably, the smoke removing apparatus 10 is installed on the application apparatus 2 and is detachable.

The tracer 110 is configured to trace the smoke, particulate matter (PM), or the vehicle exhaust gas around the user, by using image recognition technology, for example. It is of course possible to trace the user's face, hand, or other target parts. Then, the tracer 110 will accordingly generate a sensed signal, and send the sensed signal to the controller 140.

Then, the controller 140 will send a control signal to the stage 130 for the stage 130 to move the outlet 128 of the air cleaner 120, such that the outlet 128 targets at residual emitting (polluted) gas and/or the user. The stage 130 may be a stepping motor 133.

The air cleaner 120 may be a suction pump, a blower, or a fan 121, and may optionally be provided with any of the aforementioned filter components 122 or their combinations.

The outlet 128 may be formed by a plurality of holes. Moreover, the air cleaner 120 is formed with the outlet 128, and also formed with one or more additional outlets 129. The additional outlet 129 may be formed by a plurality of holes. In one embodiment, the outlet 128 and the additional outlet 129 are both configured to blow purified (clean) air toward the pollution emission source (in this case, the frying pan) and the user, so as to blow polluted air away. In another embodiment, the outlet 128 is configured to face the pollution emission source (in this case, the frying pan) to suck polluted air in and filter out the polluted air, while, the additional outlet 129 is configured to blow purified (clean) air to the user.

The power supplier 150 is used to supply power to the application apparatus 2 and the smoke removing apparatus 10 (including components therein, such as an air cleaner) of the air pollution reduction device 1.

Although the present invention has been explained in relation to its embodiments, it is to be understood that many other possible modifications and variations can be made without departing from the spirit and scope of the invention as hereinafter claimed.

What is claimed is:

1. A surgical smoke removing device, comprising a smoke removing apparatus adapted to be connected to an application apparatus, the smoke removing apparatus including:
a tracer configured to trace an emission source and/or a user's breathing zone, and obtain a sensed signal;
an air cleaner configured to provide an air column or an air curtain, and having an inlet to suck air in and an outlet to blow clean air out;
a stage configured to move the outlet of the air cleaner according to the tracer's sensed signal, such that the outlet targets at the emission source and/or the user's breathing zone; and
a controller configured to control the smoke removing device and/or the application apparatus.

2. The surgical smoke removing device of claim 1, wherein the application apparatus is a surgical lighting apparatus, a head lamp, a range hood, a flying pan, a wok, a pot, a cooker, a tea kettle, a coffeemaker, a popcorn maker, a necklace, a pair of glasses, a wristband, a watch, a mask, or an earring, which has a front side, the front side faces the emission source and/or the user's breathing zone; the outlet of the air cleaner of the smoke removing apparatus is formed as a hole or a protrusion on the front side.

3. The surgical smoke removing device of claim 1, wherein the tracer is an image recognizer, a motion sensor, or a displacement sensor, or combination thereof configured to trace a tip of a tool or a user's face, hand, or other target part; or, the tracer is a sound sensor or an ultrasound sensor, configured to detect existence of living being; or, the tracer is an infrared sensor, an optical sensor, or a visible light sensor, configured to trace a hot spot of a tool; or, the tracer is a mass sensor, a chemosensor (chemical sensor), a molecular sensor, an electronic nose, a gas chromatograph, or a mass spectrometer, or combination thereof configured to detect existence, component, or concentration of polluted air.

4. The surgical smoke removing device of claim 1, wherein the air cleaner is a suction pump, a blower, or a fan; wherein the air cleaner includes a filter component; the filter component is a high efficiency particulate air filter, an ultra-low penetration air filter, a membrane filter, a nanofiber filter, an active carbon filter, a gas filter, a catalyst filter, an antimicrobial filter, an ionizer, an ultraviolet air purifier with ultraviolet light source, an ozone generator, an impactor, or a cyclone, or combination thereof.

5. The surgical smoke removing device of claim 4, wherein the stage is an XYZ translation stage, a translation stage, a two-axis stage, a rotary stage, a tilt stage, a ball-and-socket tilt stage, or a mechanical arm, or combination thereof.

6. The surgical smoke removing device of claim 1, wherein the air cleaner further includes an additional outlet; the outlet and the additional outlet are both configured to blow purified air out or suck polluted air in, or one is configured to blow purified air out while another is configured to suck polluted air in.

7. The surgical smoke removing device of claim 6, wherein the outlet and the additional outlet are arranged such that they are both adjacent to the application apparatus, or one outlet is adjacent to the application apparatus and another outlet is far away from the application apparatus.

8. The surgical smoke removing device of claim 7, wherein the additional outlet is formed by a plurality of holes which blow air out to form the air curtain to contain pollutants, and the air curtain is located between the emission source and the user's breathing zone.

9. The surgical smoke removing device of claim 1, further comprising a power supplier configured to supply power to the application apparatus and/or the smoke removing apparatus.

10. A surgical smoke removing method, comprising:
turning on a surgical smoke removing device during a surgery process; wherein the surgical smoke removing device includes an application apparatus and a smoke removing apparatus; the smoke removing apparatus is connected to the application apparatus; the smoke removing apparatus includes a tracer, an air cleaner, a stage, and a controller; the controller is connected to the tracer, the air cleaner, and the stage;
using the tracer to trace an emission source and/or a user's breathing zone, and obtain a sensed signal;
using the stage to move the air cleaner according to the tracer's sensed signal, such that an outlet of the air cleaner targets at the emission source and/or the user's breathing zone and/or a target position; and
using the air cleaner to blow purified air to and/or suck in polluted air from one or more of the following: (i) the emission source, (ii) the user's breathing zone, and/or (iii) the target position.
